(19) United States
(12) Patent Application Publication
(10) Pub. No.: US 2021/0290179 A1
(43) Pub. Date: Sep. 23, 2021

(54) HEALTH MAPS FOR NAVIGATING A HEALTH SPACE

(71) Applicant: GOLDTECH SINO, LTD., CENTRAL, HONG KONG (CN)

(72) Inventor: EHUD BARON, OKAYAMA (JP)

(21) Appl. No.: 17/266,867

(22) PCT Filed: Aug. 7, 2019
(86) PCT No.: PCT/IB2019/056720

§ 371 Date: Feb. 8, 2021

Related U.S. Application Data
(60) Provisional application No. 62/715,325, filed on Aug. 7, 2018.

(51) Int. Cl. A61B 5/00; A61B 5/021; A61B 5/02; G16H 50/30

(52) U.S. Cl. CPC A61B 5/7275; A61B 5/021; G16H 50/30; A61B 5/7264; A61B 5/4836; A61B 5/02028

(57) ABSTRACT

A method of constructing health map based on physiological pulse shape features space of a plurality of users comprising sensing by at least one sensor a stream of pulses; extracting features and forming fuzzy clusters from the features, and constructing the health map by 2-dimensional projection of n-dimensional space.

### TECHNICAL FIELD

The present disclosed subject matter relates to visualization of health status. More particularly, the present disclosed subject matter relates to generating health space and health maps using fuzzy clustering method that can be navigated therein.

### BACKGROUND

Digital health wearables that continuously record physiological signals 24/7, generate huge amounts of valuable data. However, doctors, nurses and even users get lost in this flood of information. This is where artificial intelligence and machine learning can be used to summarize the health status changes over time in a visual intuitive display. Monitoring cardiovascular health deterioration at a glance is one of the goals of this disclosure. Several attempted to predict patient's deterioration or progress using a combined score were made; however, since the combined score is just numeric value that combines many parameters, it does not provide the multidimensional health status or how to act if immediate action is needed, when to call the doctor or what to do as first assistance.

Medicine in general, including cardiology, does not have acceptable and explicit framework that defines the position of a patient in the cardiovascular health space, her/his target zone and the paths (interventions) from the current health status to targeted health status. This makes it difficult to decide about the costs/benefits of each alternative treatment and even more, to quantify effectiveness and visualize it in a way that both the doctor and the patient can understand it.

### BRIEF SUMMARY

According to one aspect of the present subject matter, blood pressure pulse shapes can be used as indication to the cardiovascular status of the patient, and create a continuous space spanned by its N dimensional features.

It is therefore provided in accordance with an exemplary embodiment of the present subject matter a method of constructing health map based on physiological pulse shape features space of a plurality of users, the method comprising:
sensing by at least one sensor a stream of pulses;
extracting features and forming fuzzy clusters from the features; and
constructing the health map by 2-dimensional projection of n-dimensional space.

It is further provided in accordance with another preferred embodiment, a position of a specific pulse shape of a user at a specific time, indicates the health status of the user at this point in time.

It is further provided in accordance with another preferred embodiment, a plurality of positions of pulse shapes of a user overtime generates a trace that indicated changes of health status over time.

It is further provided in accordance with another preferred embodiment, the positions of pulse shapes of a user overtime generates a trace that indicates changes over time and indicates interventions such as medication, exercise, sleep, meal, toxins exposure and the like.

It is further provided in accordance with another preferred embodiment, the interventions are selected from interventions such as specific medication, specific dosage of the medication, interaction with other medications, physical rehabilitation, surgical procedure, stressful situation, life-style interventions such as exercise, sleep, healthy and unhealthy meals, glucose load, air purity, infectious disease, ionizing, and non-ionizing radiation, and the like.

It is further provided in accordance with another preferred embodiment, the health status is selected from a group of health statuses such as healthy, having health disorders, not healthy, male, female, old, young, having congestive heart failure (CHF), hypertensive, diabetic, having chronic obstructive pulmonary disease (COPD), having blood analytes disorder, electrical activity disorder, combinations of the above and the like.

It is further provided in accordance with another preferred embodiment, the method further comprising:
sensing by the at least one sensor a stream of pulses of an individual;
extracting features by decomposing sensed pulses of the individual;
classifying fuzzy clusters of the features;
estimating physiological parameters of the individual; and
placing the physiological parameters over time on the health map so as to form a personal health navigation system.

It is also provided in accordance with yet another preferred embodiment of the presented subject matter, a personal health navigation system for an individual comprising:
providing a health map as shown herein before; and
recommended paths from a user's position to a target zone on the health map.

It is further provided in accordance with another preferred embodiment, the health map comprises fuzzy clusters that are indicative of physiological parameters and therefore forms areas on the health map that are indicative of health status.

It is further provided in accordance with another preferred embodiment, the health status is selected from a group of health statuses such as healthy, sick, not healthy, male, female, old, young, having congestive heart failure (CHF), having hypertension, having diabetic, having chronic obstructive pulmonary disease (COPD), and the like.

It is further provided in accordance with another preferred embodiment, placing health parameters of the individual on the health map provides indication of a position of the individual on the map.

It is further provided in accordance with another preferred embodiment, the health map is provided with indications of healthy and unhealthy zones according to pulse features.

It is further provided in accordance with another preferred embodiment, the navigation system further comprises treatment paths indicated on the health map.

It is further provided in accordance with another preferred embodiment, the health map comprising machine learning mechanism configured to confirm with different physiological parameters of the individual.

It is further provided in accordance with another preferred embodiment, the machine learning mechanism is configured to:
estimate blood pressure from PPG signal;
determine physiological model of pulse propagation in an arterial tree;
delineate the signal to separate pulses;
use a second derivative of the PPG signal and Gaussian features indicative of the individual physiological condition.

It is further provided in accordance with another preferred embodiment, the machine learning mechanism configured to:
represent feature vectors as points in an N dimensional feature space;
construct centroid points according to the health map and the individual details selected from a group of details such as gender, age, height, weight, previous BP measurements, and the like;
perform fuzzy clustering including dimensionality reduction to improve the centroid based on recorded pulses;
position each BP pulse vector in a clustering space.

Unless otherwise defined, all technical and scientific terms used herein have the same meaning as commonly understood by one of ordinary skill in the art to which this disclosed subject matter belongs. Although methods and materials similar or equivalent to those described herein can be used in the practice or testing of the present disclosed subject matter, suitable methods and materials are described below. In case of conflict, the specification, including definitions, will control. In addition, the materials, methods, and examples are illustrative only and not intended to be limiting.

### BRIEF DESCRIPTION OF THE DRAWINGS

Some embodiments of the disclosed subject matter described, by way of example only, with reference to the accompanying drawings. With specific reference now to the drawings in detail, it is stressed that the particulars shown are by way of example and for purposes of illustrative discussion of the preferred embodiments of the present disclosed subject matter only and are presented in the cause of providing what is believed to be the most useful and readily understood description of the principles and conceptual aspects of the disclosed subject matter. In this regard, no attempt is made to show structural details of the disclosed subject matter in more detail than is necessary for a fundamental understanding of the disclosed subject matter, the description taken with the drawings making apparent to those skilled in the art how the several forms of the disclosed subject matter may be embodied in practice.

In the drawings:

FIG. 1A schematically illustrates a ring for noninvasive and unobtrusive acquisition of pulse wave needed to generate a health space and user position in it, in accordance with some exemplary embodiments of the disclosed subject matter;

FIG. 1B illustrates a perspective view of a ring for noninvasive and unobtrusive acquisition of pulse wave needed to generate a health space and user position in it, in accordance with some exemplary embodiments of the disclosed subject matter;

FIG. 2A illustrates a handheld device for noninvasive and unobtrusive acquisition of pulse wave needed to generate a health space and user position in it, in accordance with some exemplary embodiments of the disclosed subject matter;

FIG. 2B illustrates a bracelet for noninvasive and unobtrusive acquisition of pulse wave needed to generate a health space and user position in it, placed on a human wrist, in accordance with some exemplary embodiments of the disclosed subject matter;

FIG. 3 depicts a block diagram of noninvasive and unobtrusive acquisition of pulse wave needed to generate a health space and user position in it, in accordance with some exemplary embodiments of the disclosed subject matter;

FIGS. 4A-4D depict examples of raw signals received by a system, for noninvasive and unobtrusive acquisition of pulse wave needed to generate a health space and user position in it, in accordance with some exemplary embodiments of the disclosed subject matter;

FIG. 5A depicts a zoom in a noisy segment of FIG. 4C streaming pulses, in accordance with some exemplary embodiments of the disclosed subject matter;

FIG. 5B depicts a glucose spectroscopy curve, in accordance with some exemplary embodiments of the disclosed subject matter;

FIG. 5C depicts a bio impedance pulse, in accordance with some exemplary embodiments of the disclosed subject matter.

FIG. 5D depicts an intuitive Electro-Cardio-Gram (ECG) waveforms, in accordance with some exemplary embodiments of the disclosed subject matter;

FIG. 6A illustrates a superposition modeling for feature extraction, of a typical blood-pressure pulse by two Gaussian distributions, corresponding to forward and backward waves, in accordance with some exemplary embodiments of the disclosed subject matter;

FIG. 6B depicts feature extraction scheme of the superposition modeling of a blood-pressure pulse by two Gaussian distributions, in accordance with some exemplary embodiments of the disclosed subject matter;

FIG. 7A illustrates different shapes of human blood-pressure pulses corresponding to age (by decades), in accordance with some exemplary embodiments of the disclosed subject matter;

FIG. 7B schematically depicts a two-dimensional features space comprising a plurality of normalized pulse, in accordance with some exemplary embodiments of the disclosed subject matter; and

FIG. 8 is a flowchart of a method for noninvasive and unobtrusive acquisition of pulse wave needed for generating the health space and user position in it, in accordance with some exemplary embodiments of the disclosed subject matter.

FIG. 9 illustrates examples of BP pulse shapes in accordance with some exemplary embodiments of the disclosed subject matter.

FIGS. 10A-10C depict different clusters of BP pulse shapes constructed by fuzzy clustering in a BP pulse features space so as to generate fuzzy clusters around centroids, in accordance with some exemplary embodiments of the disclosed subject matter.

FIG. 11 depicts examples of mapping between BP pulse shapes and BP levels, derived from the continuous PPG in accordance to an exemplary embodiment of the present subject matter.

FIG. 12 depicts health map in accordance to an exemplary embodiment of the present subject matter.

FIG. 13 depict a health app, in which the healthy zone and unhealthy zones correspond to various diseases are indicated, in accordance with exemplary embodiments of the present subject matter.

### DETAILED DESCRIPTION

Before explaining at least one embodiment of the disclosed subject matter in detail, it is to be understood that the disclosed subject matter is not limited in its application to the details of construction and the arrangement of the components set forth in the following description or illustrated in the drawings. The disclosed subject matter is capable of other embodiments or of being practiced or carried out in various ways. Also, it is to be understood that the phraseology and terminology employed herein is for the purpose of description and should not be regarded as limiting. The drawings are generally not to scale. For clarity, non-essential elements were omitted from some of the drawings.

The terms “comprise”, “comprising”, “includes”, “including”, and “having” together with their conjugates mean “including but not limited to”. The term “consisting of” has the same meaning as “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this disclosed subject matter may be presented in a range format. It should be understood that the description in range format is merely for convenience and brevity and should not be construed as an inflexible limitation on the scope of the disclosed subject matter. Accordingly, the description of a range should be considered to have specifically disclosed all the possible sub-ranges as well as individual numerical values within that range.

It is appreciated that certain features of the disclosed subject matter, which are, for clarity, described in the context of separate embodiments, may also be provided in combination in a single embodiment. Conversely, various features of the disclosed subject matter, which are, for brevity, described in the context of a single embodiment, may also be provided separately or in any suitable sub-combination or as suitable in any other described embodiment of the disclosed subject matter. Certain features described in the context of various embodiments are not to be considered essential features of those embodiments, unless the embodiment is inoperative without those elements.

One objective of the present disclosed subject matter is to provide an easy-to-use noninvasive and continues hemodynamic monitoring (CHM) system and method that performs continuous blood pressure measurements, wherein the system can be embedded in unobtrusive wearable or a handheld device. The devices can be, but not limited to, a bracelet, a ring, a watch, an earring, a glove, a clip fastening a finger, a garment, a belt, a steering wheel, a joystick, a remote-controller, a smartphone, a mobile-phone, a tablet pc, a combination thereof, or the like. In some exemplary embodiments, the above mentioned unobtrusive wearable devices are configured to embrace a limb, such as a wrist, a finger or any human body surface where they are in close proximity to the skin of the patient' or user. For handheld devices exemplary embodiments, the blood pressure is continuously measured as long as the user is touching the handheld device.

It should be noted that, the terms “patient” or “user” are used in the present disclosed subject matter to indicate any individual subject, such as an athlete, a patient, an active person, an elderly person, or any person can wear or hold the noninvasive CHM device for hemodynamic monitoring.

The system and method as described in this disclosure teach the estimate of continuous hemodynamic parameters by non-invasively acquiring a stream of blood-pressure pulses, process and analyze the pulses to estimate blood-pressure and other hemodynamic parameters. The CHM system and method manifest a superior alternative to the automated BP measurements based on the oscillometric method, used since 1876, and provides continuous monitoring rather than intermittent monitoring.

In some exemplary embodiments, the CHM system comprises transmissive and or reflective optical sensor; Bio-impedance sensor; ECG sensors, a combination thereof, or the like for acquiring signals representing a physiological condition of a patient. Additionally, or alternatively, in some exemplary embodiments, the CHM system comprises a concave optical PPG sensor and a pressure or force sensor. Typically, optical sensors are placed in an optimal place for optical sensor, in a place that provides good quality continuous PPG signal, while pressure or force sensors are placed above the radial artery.

One technical problem dealt with by the disclosed subject matter is that large number of data points accumulated for the evaluation of hemodynamic parameters is used in commercially available deep learning concepts, don't necessarily resolve the relationship between the input and the output for any given patient. Such relationship is based on very large number of statistical data, which results in lengthily and cumbersome computations and doesn't clarify physiological occurrences. Moreover, products utilizing such methods Nd to overlook most of the data and sacrifice accuracy for speeding up the calculation. Although deep learning based on large statistical data is mostly correct, it is not correct for all patients despites the lengthily process.

Another technical problem dealt with by the disclosed subject matter is propagation characteristics of arteries. Clearly, arterial pressure pulse changes while traveling away from the heart towards the peripheral arteries. These changes affect the mean and diastolic pressure, however not the systolic pressure. Thus, the relationship between central and peripheral pulse pressure depend on propagation characteristics of the arteries. While, the sphygmomanometer gives values of the systolic and diastolic pressure, the CHM system of the present discloser can obtain additional information from the time-varying pulse waveform that improve quantification of the systolic load on the heart and other central organs.

One technical solution of the CHM system, implemented by a system on a chip (SoC), is a method based on a model utilizing adaptive machine learning that requires fewer data points. Thus, the method taught in this disclosure rapidly and accurately converge sampling to relevant continues monitoring for each individual. Furthermore, the method improves the accuracy of the results over time usage. It will be noted that although the embodiment described below are focused on hemodynamic monitoring, the disclosed subject matter can be employed to measure and monitor other physiological parameters.

Another technical solution provided in the present disclosure is acquiring a signal comprised of stream of blood pressure pulses with one or more optical sensors, bio-impedance sensors, a combination thereof, or the like. The blood-pressure sensing is followed by utilizing a unique algorithm for real time analysis of the sensor's signals in order to determine a patient's continuous blood-pressure and hemodynamic parameters. Each pulse is approximated by a model pulse that consists of at least two components, the forward moving wave and reflected waves. The forward moving wave component is generated by the ejection of blood from the left ventricle of the heart. The reflected waves are the result of interaction between the forward moving component of the arterial tree and the capillary system.

One advantage of utilizing the noninvasive CHM system and method of the present disclosure is eliminating the need of skilled medical personnel for CHM procedure. The noninvasive system is suitable both for hospitals and wellness and its usage as wearable consumer product requires no medical background.

Another advantage of utilizing the noninvasive CHM system and method of the present disclosure is reducing complications, unpleasantness, and pain as well as hospitalization cost.

Yet another advantage of utilizing the noninvasive CHM system and method of the present disclosure is measurement accuracy comparable with invasive methods.

It should be noted that blood-pressure pulses are composed of a forward moving component and a reflected component. The forward moving component is generated by the ejection of blood from the left ventricle of the heart and the reflected component results from interaction between the forward component and the arterial tree and capillary system. Each pulse may be characterized according to a model of a pulse comprising at least two partly overlap Gaussian functions that have different peaks and spread. Accordingly, the forward moving component and the reflected component can each be represented by one of the at least two Gaussian functions.

It will be noted that the method disclosed herein utilizes passive modalities, active modalities, a combination thereof, or the like for acquiring blood-pressure and other hemodynamic signals. The active modalities can be differentiated from one another by either their sensor technology and/or the way it is activated. In some exemplary embodiments, active modalities can be based on a plurality of sensors comprising bio impedance sensors, Tonometric blood-pressure device, and optical sensors such as: PhotoPlethysmoGraph (PPG) sensors and spectroscopic sensors. The passive modalities can be electrocardiogram (ECG) sensors, or the like

It should also be noted that signals (sequence of hemodynamic pulses) acquired from the previously described sensors or devices, contain noise and other artifacts. Thus, not all the acquired pulses can contribute for analyzing hemodynamic parameters of a patient. Hemodynamic pulses, such as blood-pressure and ECG pulses are well known in the art and may be characterized by a pair of Gaussian functions representing a hemodynamic pulse baseline (typical pulse). In some exemplary embodiments, such base line can be used to filter out noisy pulses and or signal portions that can't contribute to the pulse analysis. Additionally, or alternatively, the filter baseline parameters can be offset according to the patient specific data, such as demographic and precondition information.

In some exemplary embodiments, the CHM system of the present disclosure can utilize fuzzy clustering to determine a centroid pulse that can later be synthesized from the two Gaussian superposition. This synthesized pulse best represent the pulse sequence and can be used to determine the hemodynamic parameters.

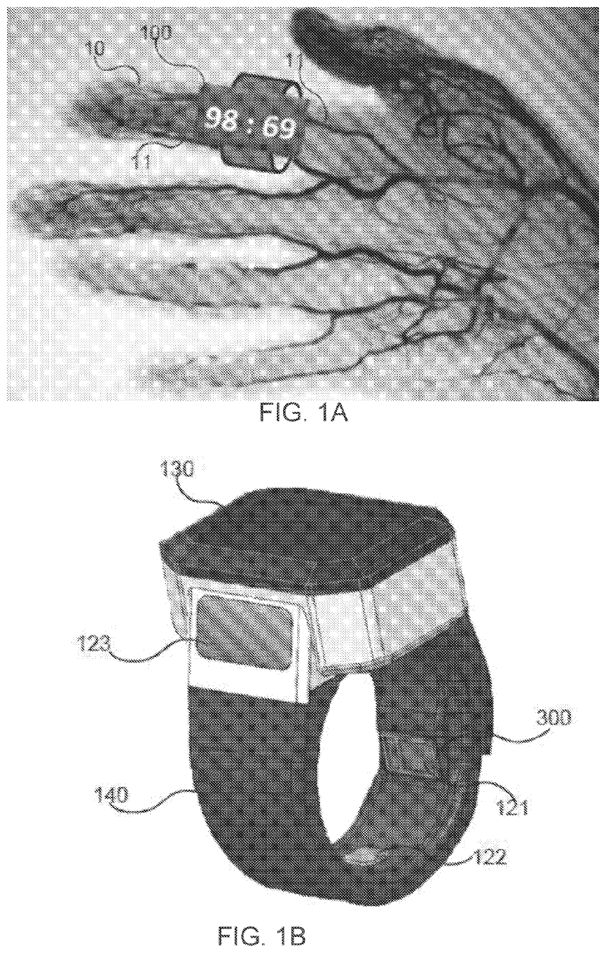

Referring now to FIG. 1A schematically illustrating a ring for noninvasive and unobtrusive acquisition of pulse wave, needed for generating the health space and user position in it, placed on an imaged human finger, in accordance with some exemplary embodiments of the disclosed subject matter. A ring 100 provided with a system for noninvasive CHM is placed on an index finger 10. It should be noted that the ring 100 is a preferred embodiment however, a plurality of unobtrusive wearable articles can be utilized for embodying the noninvasive CHM. In some exemplary embodiments, unobtrusive wearable articles can be a belt-like element configured to embrace a patient's limb, e.g., a wrist, in areas where arteries are in close proximity to the patient's skin. The wearable articles can be for example a bracelet, a hand-watch, a ring, or the like.

The ring 100 is shown to be placed on the index finger 10, wherein the ring is in close proximity to the arteries 11 that are being digitally represented. The ring is placed on the finger in a noninvasive manner.

Referring now to FIG. 1B illustrating a perspective view of a ring for noninvasive and unobtrusive acquisition of pulse wave, needed for generating the health space and user position in it, in accordance with some exemplary embodiments of the disclosed subject matter. The ring 100 comprises at least one optical sensor 121, as at least one bio impedance sensor 122, as at least one ECG sensor 123, a CHM system on a chip (SoC) 300, a display 130, a illumination LEDs (Red/Ir) on other side 140, a combination thereof, or the like. The ring 100 is configured to fit on human fingers. The form factor of ring 100 can be adapted to achieve good coupling between finger 10 and the sensors provided to the ring 100. For example, a pressure sensor of the ring 100 can be situated in an optimal proximity to the artery of the finger (not shown in this figure).

It should be noted that all the components of ring 100 can be interconnected with each other by wires within the ring, particularly with SoC 300, which shall be discussed in details below.

In some exemplary embodiments, the system and method of the disclosed subject matter can utilize a plurality of sensors, such as PhotoPlethysmoGraph (PPG) sensors, Tonometric sensors, electrocardiogram (ECG) sensor, a combination thereof, or the like.

In some exemplary embodiments, the optical sensor 121 can be utilized for PhotoPlethysmoGraph (PPG), i.e., optically obtained volumetric measurement of an organ. Optical sensor 121 can comprise at least one photo-diode and at least one light emitting diode (LED). The PPG can be obtained by the photo-diode, which measures changes in light absorption of an organ that was illuminated by the LED.

In some exemplary embodiments, the optical sensor 121 can be an optical transmissive or an optical reflective sensor, an optical concave sensor, a combination thereof, or the like. Additionally, or alternatively, a spectroscopic modality can be utilized for acquiring pulses needed for estimating the continuous blood-pressure. In such modality, the optical sensor 121 comprises a plurality of wave length light sources (LEDs) and a plurality of wavelength receivers (photo diodes).

It should be noted that the sensors in the ring 100 can be used in various ways. As an example, the ring 100 can be configured for continuous estimation of blood-pressure; cardiac output; stroke oxygen saturation in peripheral blood (SpO2), from the red and infrared PPG signal; a combination thereof, or the like. It should also be noted that although principles (PPG signal) similar to commercially available oximeters can be employed, the present disclosure surpass oximeters by focusing, in addition, on changes of absorbance of red and infrared light during peak and through of the red and infrared representative synthesized pulse.

In some exemplary embodiments, the bio impedance sensor 122 can be utilized as impedance plethysmograph for measuring changes in volume within the finger blood vessel in order to determine circulatory capacity or cardiac output. Additionally, or alternatively, the bio impedance sensor 122 can be used as a complimentary electrode for ECG measurements.

In some exemplary embodiments, the ECG sensor 123 can be electrode activated by touching it with a finger of a contra-lateral hand, wherein the other electrode is the bio impedance sensor 122.

In some exemplary embodiments, the display 130 can be an alphanumeric or bitmap, LED display, liquid crystal display (LCD), a combination thereof, or the like. Display 130 can be wired to the SoC 300 and configured to display the SoC 300 outcome results, such as intermittent/continuous systolic/diastolic pressure, mean arterial pressure SPo2, heart rate, stroke volume, cardiac output, a combination thereof, or the like.

In some exemplary embodiments, the antenna 140 is embedded within ring 100. Antenna 140 can be mutually coupled with a transceiver of the SoC 300 for communicating with Bluetooth and/or Wi-Fi devices.

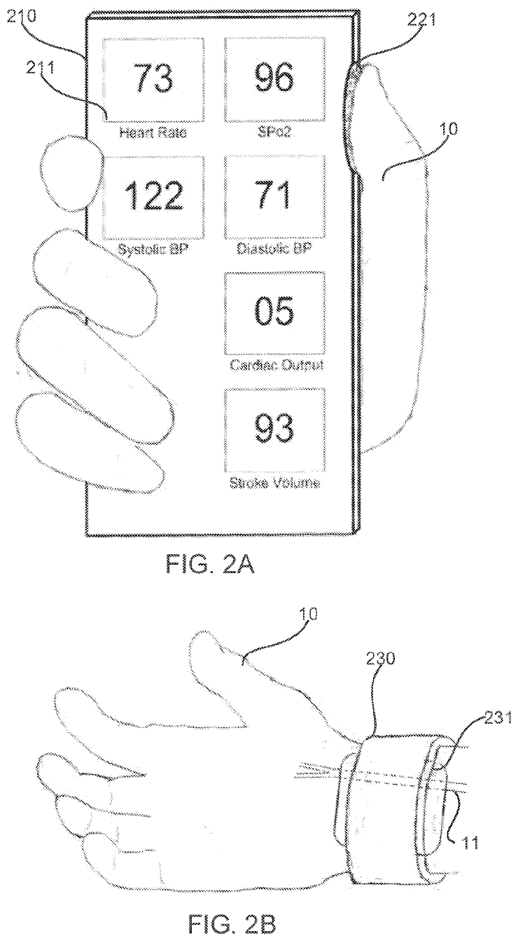

Referring now to FIG. 2A illustrating a handheld device for noninvasive and unobtrusive acquisition of pulse wave, needed for generating the health space and user position in it, in accordance with some exemplary embodiments of the disclosed subject matter. The handheld device 200 comprises a noninvasive touchpad 221 and a display 211, a combination thereof, or the like.

In some exemplary embodiments, handheld device 210 can be a cellular phone, phone handset, a smartphone, a handheld remote controller, an electronic device handle, a joystick, a car steering wheel, a tablet, a notebook pc, a combination thereof, or the like. It will be noted that in some embodiments, display 211 can be an integral part of the handheld device 210 and can be utilized to display hemodynamic information. For example: heart rate, SPo2, systolic and diastolic blood-pressure, cardiac output, stroke volume, a combination thereof, or the like.

In some exemplary embodiments of the disclosed subject matter, the noninvasive touchpad 221 can be embedded in a handheld device 210, as an example: Samsung Galaxy smartphone. Touchpad 221 is configured to sense the user's blood-pressure pulse as long as the user's finger 10 is places on touchpad 221. The touchpad 221 comprises an optical sensor, a reflective optical sensor or concave optical sensor, a combination thereof, or the like.

Additionally, or alternatively, the noninvasive touchpad 221 further comprises a bio impedance sensor (not shown), an ECG sensor (not shown), an SOC (not shown), such as SoC 300 and a combination thereof. In some exemplary embodiments, touchpad 221 can be directly wired to the processor of the handheld device 210 in order to perform operations required to continuously monitor the patient's hemodynamic condition.

It should be understood that the user skin has to touch the non-invasive wearable articles of the embodiments described in the present disclosed subject matter.

Referring now to FIG. 2B illustrating a bracelet for noninvasive and unobtrusive acquisition of pulse wave, needed for generating the health space and user position in it. placed on a human wrist, in accordance with some exemplary embodiments of the disclosed subject matter. Bracelet 230 comprises a topometric sensor (not shown in the figure) embedded within inflatable bladder 231 provided in the inner side of the bracelet. Bracelet 230 further comprises sensors (not shown) similar to the sensors used in ring 100. Furthermore, bracelet 230 comprises components (not shown) such as CHM system on a chip (SoC) as will be discussed later on, a display, an antenna, such as of ring 100 as depicted in FIG. 1B. It should be noted that bracelet 230 can be configured to perform processes and outcomes similar or identical to the processes and outcomes provided by ring 100, thus estimating intermittent or continuous of systolic/diastolic pressure, mean arterial pressure SPo2, heart rate, stroke volume, cardiac output, a combination thereof, or the like.

In some exemplary embodiments, the bracelet 230, provided with Tonometric sensor, further comprises a strap configured to fasten the bracelet for non-invasive continuous blood pressure monitoring to the limb, preferably a wrist, in a manner that holds the bracelet 230 and the inflatable bladder 231 stable against palpable artery such as radial artery 11, when the inflatable bladder 231 is inflated. The bracelet form factor can be configured to achieve good coupling between the radial artery and pressure sensor/actuator using Tonometric concept to estimate continues blood-pressure.

Bracelet 230 can be provided with a CHM system controller, such as SoC 300 of FIG. 1B, to acquire the blood-pressure pulses by non-invasively manipulating the air-filled bladder 231 placed on radial artery 11 to sense the pressure of the artery and acquire blood-pressure pulses. Subsequently, the controller may perform computations required to provide the estimated CHM information.

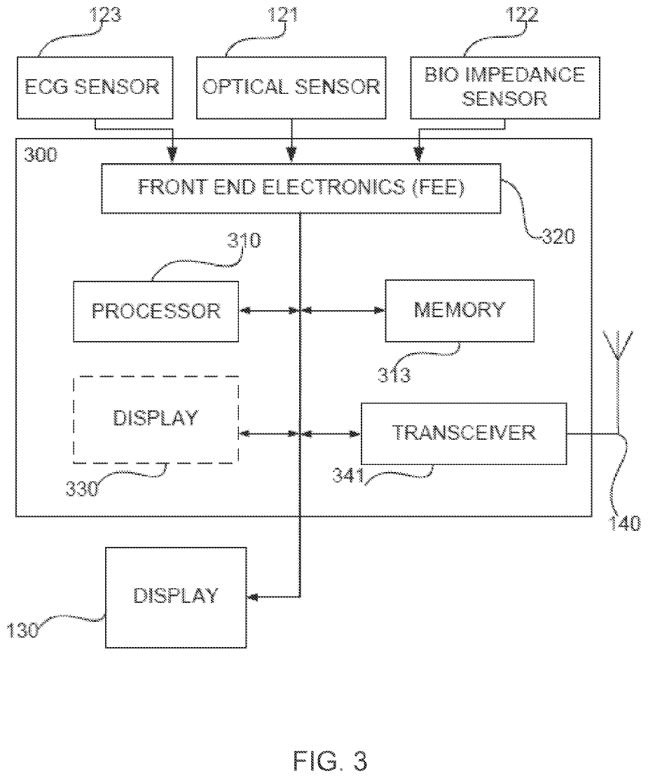

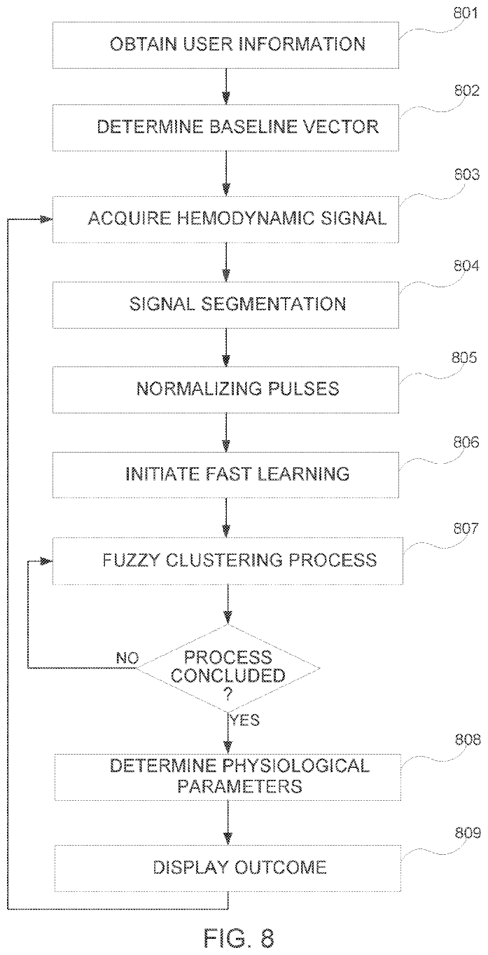

Referring now to FIG. 3 depicting a block diagram of a for noninvasive and unobtrusive acquisition of pulse wave, needed for generating the health space and user position in it. system, in accordance with some exemplary embodiments of the disclosed subject matter. SoC 300 can be a computerized component adapted to acquire and process hemodynamic signals. In some exemplary embodiments, SoC 300 and any of its subcomponents can be incorporated on a single microelectronic chip dedicated for performing methods, such as depicted in FIG. 8, for real-time determination of hemodynamic information.

In some exemplary embodiments, the SoC 300 comprises a processor 310. The processor 310 can be a central processing unit (CPU), a microprocessor, a field-programmable gate array (FPGA), an application-specific integrated circuit (ASIC), an electronic circuit comprising a plurality of integrated circuits (IC), a combination thereof, or the like. The processor 310 can be used to perform real-time signal processing of hemodynamic sensors, and computations required by the SoC 300 or any of its subcomponents to determine hemodynamic parameters/data of the patient.

In some exemplary embodiments of the disclosed subject matter, SoC 300 comprises front-end electronics (FFE) 320. FEE 320 can be used to acquire data from sensors that are situated in close proximity to a patient's skin. These sensors yield analog signals that are indicative of human hemodynamic pulses; the sensors can be sensors such as optical sensor 121; bio impedance sensor 122; ECG sensor 123, of FIG. 1B; noninvasive touchpad 221, of FIG. 1B; a combination thereof, or the like.

FFE 320 can be an electronic circuit comprising a plurality of integrated circuits (IC), such as analog-to-digital converter (ADC), noise filters, amplifiers, a combination thereof, or the like. In some exemplary embodiments, FFE 320 can be used for preprocessing the raw signal in order to remove noisy segments and isolate segments suitable for analysis. Noise, spikes and motion artifacts in the raw signals typical results from motion of the sensor relative to the skin. Upon filtering-out noises from raw signals, the FFE 320 shape these analog signals prior to converting them to digital representation by the FEE'S ADC.

The digital representation of the sensors may be retained by processor 310 as sensors raw data. Additionally, or alternatively, the FEE 320 can modulate optical sensor's light to frequency signal and then communicate the signal to processor 310.

In some exemplary embodiments, the processor 310 can communicate outcomes of real-time CHM and/or stored CHM parameters to be displayed on displays, such as display 130, which can also be seen in FIG. 1B. In the exemplary embodiments, where display 130 is an integral part of a wearable device, the display 130 can be wired to the SoC 300 for communicating the outcomes, wherein the wires are embedded within the device. In other exemplary embodiments, the SoC 300 comprises display 330. Display 330 can be incorporated within the SoC 300 and configured to carry out functions equivalent to display 130.

It should be noted that the outcome of the SoC 300 can be alphanumeric information depicting the hemodynamic parameters of the patient/user. As an example, the outcome comprises systolic/diastolic pressure, mean arterial pressure SPo2, heart rate, stroke volume, cardiac output, a combination thereof, or the like.

Additionally, or alternatively, SoC 300 comprises a transceiver 341. Transceiver 341 can be used to provide an interface for wireless communication of hemodynamic parameters outcomes to external devices, such as a PC, display devices, a smartphone, a tablet pc, a hospital monitor, the Internet, or the like. Transceiver 341 can use wireless communication technologies; such as Bluetooth, Wi-Fi, a combination thereof, or the like, to transmit outcomes and receive instructions over antenna 140 (also shown in FIG. 1B).

In some exemplary embodiments of the disclosed subject matter, instructions transmitted from a medical center, doctor's office or the like, may be received by transceiver 341 and processed by processor 310. The instructions can comprise, but not limited to, system activation, system hibernation, retrieval of past hemodynamic data, selection of required hemodynamic parameter, a combination thereof, or the like. It should be noted that communication of hemodynamic parameters outcomes to the Internet can be used to transmit outcomes to the cloud for patient monitoring.

In some exemplary embodiments, the SoC 300 comprises a memory 313. Memory 313 can be a hard disk drive, a flash disk, a random-access memory (RAM), a memory chip, a flash memory, a combination thereof, or the like. In some exemplary embodiments, memory 313 can be used to retain software elements, data elements, a combination thereof, or the like. The software elements can comprise algorithms, programs, instructions, functions, and files that are operative to cause processor 310 to perform methods, such as depicted in FIG. 8 as well as acts associated with SoC 300 and its subcomponents. In some exemplary embodiments, the data elements are a memory module comprising the sensors raw data and outcomes of CHM, a combination thereof, or the like.

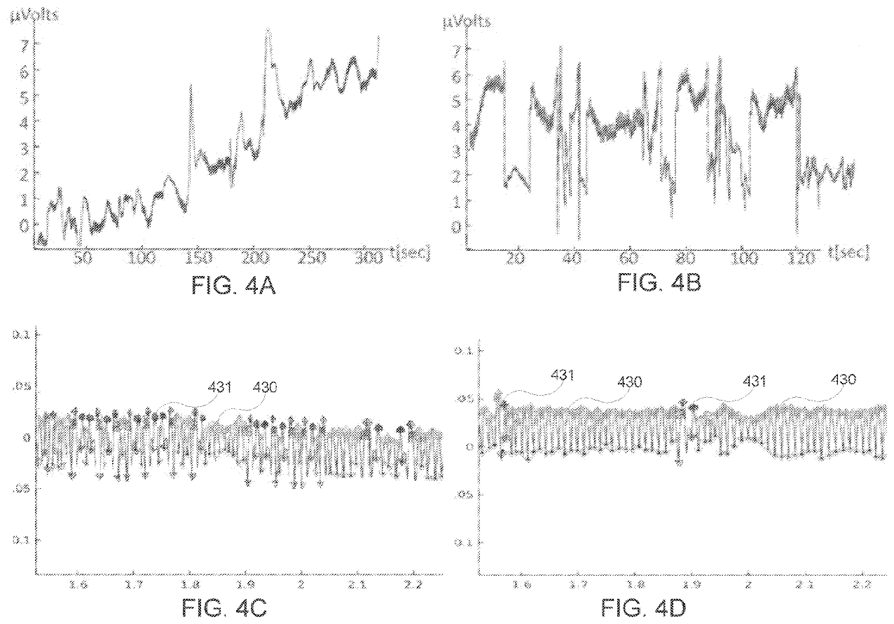

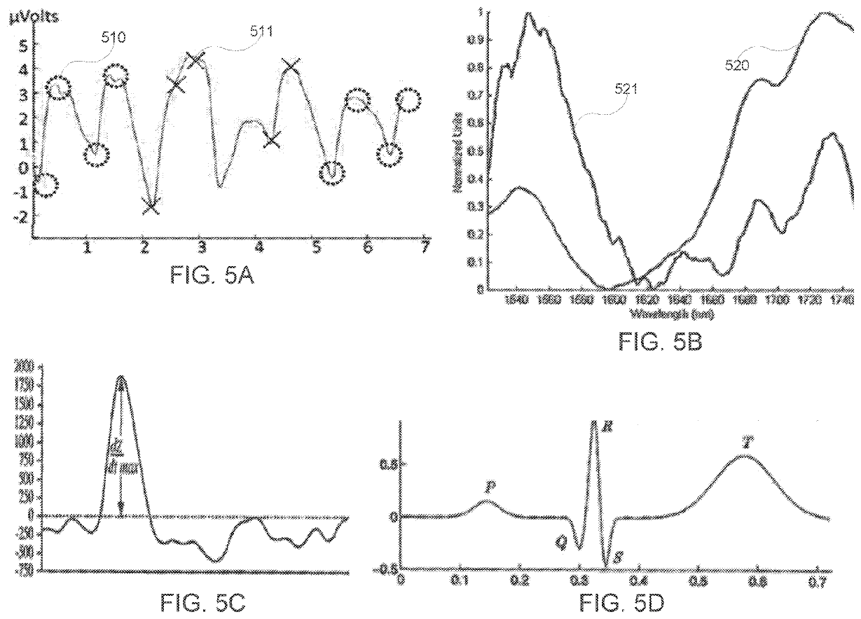

Referring now to FIGS. 4A, 4B, 4C, 4D and 5A depicting examples of raw signals received by a for noninvasive and unobtrusive acquisition of pulse wave, needed for generating the health space and user position in it. system, in accordance with some exemplary embodiments of the disclosed subject matter and a zoom in a noisy segment of FIG. 4C streaming pulses, respectively. The raw signals depicted in FIGS. 4A to 4D and 5A represent pulses of stream of blood-pressure acquired from sensors, such as optical sensor 121, of FIG. 1B; noninvasive touchpad 221, of FIG. 2A; an inflatable bladder 231, of FIG. 2B; a combination thereof, or the like.

Typically, the raw signal comprises high frequency noise and spikes resulting from motion artifacts caused by movement of the sensors relative to the skin. In some exemplary embodiments, the FFE 320, of FIG. 3 is used for preprocessing of the raw signal is done in order to remove extremely noisy segments and find segments that carry relatively better-quality signal. It should be noted that a noise free blood-pressure pulse last approximately one second and has amplitude of few micro volts. In some embodiments, FFE 320 can amplify the raw signal for further processing.

The noisy raw signal shown in FIG. 4A is an example of unacceptable signal, having duration of 300 seconds and a significant DC component drift. In the example of FIG. 4B, the raw signal has 120 seconds duration and a stable DC; however, its AC component is unstable, due to motion artifacts.

FIG. 4C depicts a segment of streaming pulses having a significant amount of pulses corrupted by noise 431 (marked black) and a small amount of acceptable pulses (marked gray) 430, which indicate that the pulses are mostly unacceptable for analysis. The distinction between acceptable and unacceptable pulses may be done using a threshold level to simplify processing. FIG. 4D depicts a segment of streaming pulses having positive peaks marked gray, which indicate that the pulses are mostly acceptable for analysis. FIG. 5A is depicting a zoom-in of a noisy segment of FIG. 4C streaming pulses. The pulses marked with a circle 510 are acceptable pulses while pulses marked with an X 511 are corrupted pulses.

It should be noted that the unacceptable pulses described above may fall far away from the centroid of the fuzzy clustering (to be described in detail herein after) and thus will have an insignificant impact on the determined centroid.

Referring now to FIG. 5B depicting glucose spectroscopy curve, in accordance with some exemplary embodiments of the disclosed subject matter. The information of the glucose spectroscopy curve can be acquired by sensors, such as optical sensor 121; of FIG. 3. In some exemplary embodiments, the spectroscopy presentation comprising principal component loading 520 and pure component glucose spectrum 521 can be used for learning glucose concentration in blood from glucose spectroscopy.

Referring now to FIG. 5C depicting examples of raw signals obtained by a bio impedance sensor, in accordance with some exemplary embodiments of the disclosed subject matter. In some exemplary embodiments, physiological parameters such as cardiac output estimation, stroke volume, blood-pressure, and a combination thereof; can be computed from analyzing bio-impedance signals that are received from bio impedance sensor such as sensor 122 shown in FIG. 3.

Referring now to FIG. 5D depicting examples of an Electro-Cardio-Gram (ECG) waveforms, in accordance with some exemplary embodiments of the disclosed subject matter. The ECG waveform can be obtained from an ECG sensor 123 shown in FIG. 3, as an example.

It should be noted that the raw signals acquired by different sensors as depicted above can be modeled by a variety of methods in order to extract the physiological parameters for CHM. One exemplary method of modeling the pulses is based on calculating the peaks of the second derivative of the acquired pulses. Another exemplary method of modeling the pulses is based on superposition of at least two Gaussian curves corresponding, for example, to the forward and backward waves. Since both models are known in the art and for the sake of simplicity, the present disclosure focuses on the use of Gaussian model for extracting data points required for the described method.

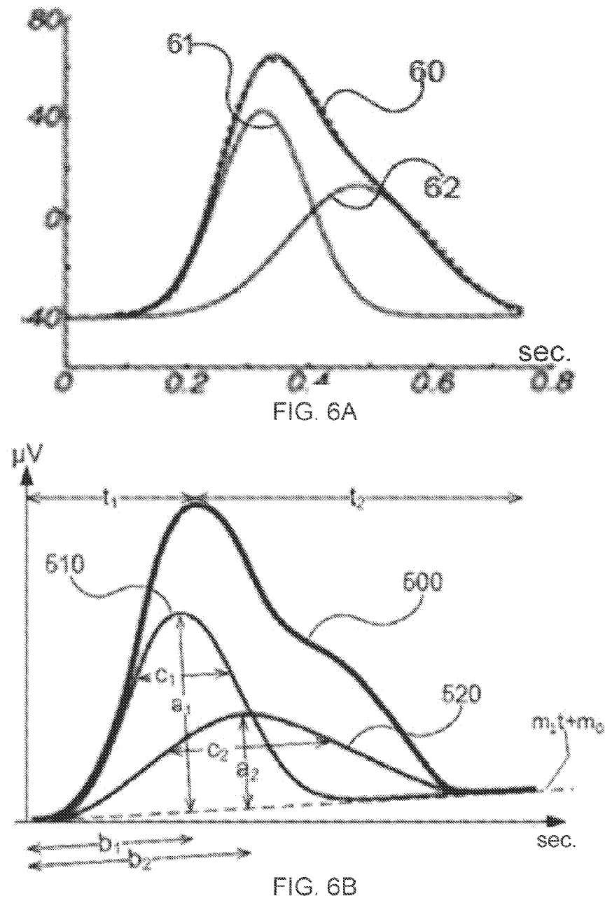

Referring now to FIG. 6A illustrating a superposition modeling of a typical blood-pressure pulse by two Gaussian distributions, corresponding to forward and backward waves, in accordance with some exemplary embodiments of the disclosed subject matter.

A forward going wave represented by curve 61 is generated by the ejection of blood by the left ventricle representing the diastolic part of the blood-pressure pulse. A backward wave represented by curve 62 is caused by reflection from the arterial blood pressure with main reflection from the iliac bifurcation. In some exemplary embodiments, pulse 60 is acquired by sensors as disclosed herein.

Referring now to FIG. 6B depicting features scheme of the superposition modeling of a normalized pulse by two Gaussian curves, in accordance with some exemplary embodiments of the disclosed subject matter.

The parameters scheme of the superposition modeling of a normalized pulse 600 uses a mathematical model of synthesizing a pulse shape similar to blood-pressure pulses acquired by the sensors detailed in the present disclosure.

The forward going wave is generated by the ejection of blood by the left ventricle is modeled (normalized) mathematically as a first Gaussian curve (1GC) 610 representing the diastolic part of the blood-pressure pulse. The backward wave caused by reflection from the arterial systems with main reflection from the iliac bifurcation is normalized mathematically as a second Gaussian curve (2GC) 620 which is smaller than 1GC 610 and is shifted in time. The Gaussian mathematical model can comprise at least the following features:
[t₁] indicates the time duration from pulse onset to systolic peak.
[t₂] indicates the time duration from systolic peak to pulse end.
[a₁] indicates the 1GC 610 peak amplitude.
[a₂] indicates the 2GC 620 peak amplitude
[b₁] indicates the 1GC 611 rise time with respect to t₁
[b₂] indicates the 2GC 621 rise time with respect to t₁
[c₁] indicates the 1GC 612 time duration spread.
[c₂] indicates the 2GC 622 time duration spread.
[m₁] indicates the DC component slope.
[m₀] indicates the DC component bias.

It should be noted that based on heart pumping model, 1GC 610 and 2GC 620, representing systolic and reflected waves respectively, have a physiological parameter that correspond to the listed above features. For example: a₁ corresponds to the central blood-pressure systolic pressure; c₁ corresponds to the left ventricle volume multiplied by ejection fraction; c₂ represents the reflected wave and the systemic vascular resistance; b₂-b₁ is proportional to pulse wave velocity in the aorta, where main reflection is from the iliac bifurcation.

In some exemplary embodiments, each normalized pulse can be calculated according to the following equation:

\tilde{x}ₙ(t)=Γ(\tilde{W̅}[n],t)+, t∈X, where Γ(\tilde{W̅},t) is a curve defined by the parameter vector \tilde{W̅}, \tilde{r}(t) is the residue, and T≡[0,η_{T1}+η_{T2}] is the normalized time domain in which pulses are defined, n=1 . . . N is the beat counter. Since the pulses in the original signal are due to the superposition of the forward and the backward wave, the following model, consisting of the summation of two Gaussian curves (SoGj), can be used:

$$y ( t ) = {a}_{1} exp [ - \frac{{( t - {b}_{1} )}^{2}}{{c}_{1}^{2}} ] + {a}_{2} exp [ - \frac{{( t - {b}_{2} )}^{2}}{{c}_{2}^{2}} ]$$

Where the parameters are defined as follows:
aᵢ model the amplitude of the curves;
bᵢ is the location of their maxima; and
cᵢ represents their width.

In some exemplary embodiments, y(t) represents, at any given time of the pulse, the value of a normalized pulse 600 that represents an actual hemodynamic pulse as represented by GC 60. It will be noted that, for extracting the at least N features of each pulse, the calculation of y(t) can be repeated n times for each pulse 60 duration. Based on the repeated calculation of y(t), the SoC 300 shown in FIG. 3 is determining a N-dimensional vector that represents the normalized pulse 600. Thus, the SoC 300 can sample the pulse 60 every predetermined time (δt). As an example, if the pulse duration is 1200 milliseconds and (δt) equals 4 milliseconds, hence (n) equals 300 samples. It should be understood that y(t) is calculated for each (δt) and the accuracy of the N-dimensional vector, representing normalized pulses 600, is directly proportional to (n). The number of streaming pulses obtained from a raw signal, per each study, may vary in accordance with the specific examined patient/user activity condition. Such condition can be resting, sleeping, exercising, preexisting medical status, steady state, a combination thereof, or the like. In some exemplary embodiments, the number of streaming pulses per study (a) can vary between 10 to 40 pulses per study; also, an outcome detailing the user hemodynamic parameters can be displayed for each study. It will be understood that the studies may be repeated indefinitely, which subsequently improve the accuracy of hemodynamic parameters estimation.

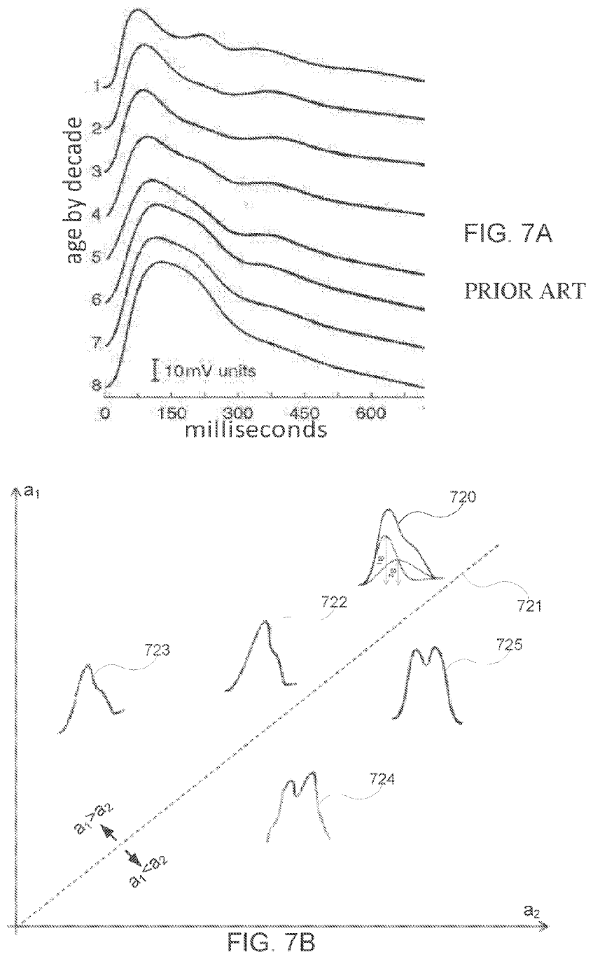

Referring now to FIG. 7A illustrating different shapes of human blood-pressure pulses corresponding to age (by decades), in accordance with some exemplary embodiments of the disclosed subject matter. The graphs in this figure, resulting from statistical analysis of blood-pressure pulse, clearly manifest the significant changes of the pulses along human's life cycle. Additionally, or alternatively, such statistical analysis can be made per year, per gender, per height, per weight, a combination thereof, or the like. For each graph, showing pulse shape in the brachial artery that changes with age, the superposition modeling as shown in FIG. 6B can be implemented. Furthermore, the statistical analysis can be tuned to comprise additional profiling information, such as exact age, gender, height, weight, medical precondition, a combination thereof, or the like. Given the above, a N-feature vector can be determined for each profile, thus each user may be provided with a baseline of the N-feature vector (baseline vector) according to the profile he or she matches.

Referring now FIG. 7B schematically depicting a two-dimensional features space comprising a plurality of normalized pulse, in accordance with some exemplary embodiments of the disclosed subject matter. FIG. 7B depicts two dimensions for illustration purposes only but these two dimensions are taken out of the at least N-dimensional space, which represent the features discussed herein before. In this particular example, the two dimensions are a₁ that indicates a first GC peak amplitude, and a₂ that indicates second GC peak amplitude. In some exemplary embodiments, each sampled actual pulse, substituted by normalized pulse, can be represented by N-feature vector. The N dimensions space can be implemented as a cluster allocated to store a plurality of N-feature vectors of acquired pulses, wherein the cluster can be a part of a memory such as memory 313 of FIG. 3, configured to store N-feature vectors. The maximum amount of the plurality of N-feature vectors is equal to the number of streaming pulses per study (σ) plus the baseline vector (σ+1).

In some exemplary embodiments, vector 720 can be a baseline vector indicating a most suitable pulse for the user. Vector 722 is an example indicating an allowable pulse, where the ratio between a₁ and a₂ is typical to old age. Vectors 724 and 725 are examples indicating non-allowable pulses. Vector 723 is an example indicating an allowable pulse, having relatively smaller amplitude that also corresponds to older physiological age. Additionally, or alternatively, diagonal 721 behaves as a filter for corrupted pulses. All pulses falling below the diagonal 721, pulses having a₁<a₂, can be discarded since by definition a₁ representing the amplitude systolic blood-pressure must be greater than a₂ representing diastolic blood-pressure. In some exemplary embodiments, similar type of analyzing relationships of the features can be utilized to filter out corrupted pulses.

In some exemplary embodiments, upon populating the N-dimension cluster, vector 720, i.e. baseline vector, can initially be nominated as the centroid of the cluster.

Referring now to FIG. 8 that represents a flowchart of a method for noninvasive and unobtrusive acquisition of pulse wave needed for generating the health space and user position in it, in accordance with some exemplary embodiments of the disclosed subject matter.

In step 801, the user's information is obtained. In some exemplary embodiments, the user's information comprises profiling information, such as age, gender, height, weight, medical precondition, blood pressure, and heart rate on record, type of measurements on record, a combination thereof or the like.

In step 802, a baseline vector is determined. In some exemplary embodiments, the baseline vector can be based on the at least N features vector of the profile that the fits the user. Additionally, or alternatively, the baseline vector can be used as a centroid of the initial fuzzy clustering process, to be describe in detail herein after.

In step 803, at least one signal from the plurality of hemodynamic sensors is acquired. The sensors comprise bio impedance sensors, Tonometric pressure sensors, PPG optical sensors, spectroscopic multi-wavelength optical sensors, and ECG biopotential sensors, a combination thereof, or the like.

In step 804, the at least one hemodynamic signal can be segmented. In some exemplary embodiments, the FEE 320 of SoC 300 (shown in FIG. 3) can be used to filter out noisy segments of the raw signal (such as the segment depicted in FIG. 4C), where the signal quality index is below a certain threshold. Furthermore, the remaining can be divided into pulses.

In step 805, the pulses are normalized. In some exemplary embodiments, the normalizing can be based on a Gaussian distributions model comprising 1GC 610 and 2GC 620, as shown in FIG. 6A for example. Furthermore, the at least N features, i.e. pulse vector, of the pulse is determined.

In step 806, fast learning is initiated. In some exemplary embodiments, the fast learning is initialized upon populating the baseline vector and the plurality of normalized pulses vectors, wherein the amount of the plurality of normalized pulses (each having at least N features) is given by the number of streaming pulses per given study σ. In this initial step, the baseline vector is designated as the centroid of the cluster.

In step 807, a fuzzy clustering process is executed. In some exemplary embodiments, the fuzzy clustering process first centroid of the cluster adopts the values of the baseline vector. Then, the centroid is learned as the amount of the plurality of normalized pulses in cluster growth. The centroid is a weighted average of the neighboring normalized pulses in the 10-dimensional space, wherein the weight of each normalized pulse in the cluster is inversely proportional to its distance from the centroid. Thus, the further away a pulse is, the less is its influence on the centroid.

The fuzzy clustering and formation of centroid can be performed by the following pseudo code:
I. Set up a fuzzy partition u(⋅) of k nonempty membership functions, u(⋅)≠0, 1≤i≤k and 2≤k≤n, wherein n is the number of elements in the data set X.
II. The k weighted means are calculated by using the following formula:

$${v}_{i} = {\sum }_{x ∈ X} {( {u}_{i} ( x ) )}^{2} * \frac{x}{{{Σ}_{x ∈ X} ( u i ( x ) )}^{2}} , 1 \leq i \leq k and x ∈ X$$

III. A new partition, u(⋅) is constructed as follows: let I(x)={1≤i≤k|vᵢ=x}=0, put:

$${\hat{u}}_{j} ( x ) = {\sum }_{i = 1}^{k} \frac{1 / {‖ x - {v}_{j} ‖}^{2}}{1 / {‖ x - {v}_{i} ‖}^{2}}$$

else let î be the least integer in I(x) and put:

$${\hat{u}}_{i} ( x ) = \{ \begin{matrix}1 if i = î \\ 0 if i \neq î\end{matrix} for 1 \leq i \leq k$$

IV. If the difference between u(⋅) and û(⋅) is less than a specific threshold value, T, then stop; otherwise, set u(⋅) to û(⋅) and go to step II.

Following the fuzzy clustering process, the centroid can move, in the N-dimensional space, to reflect contribution from the neighboring normalized pulses and thus, become a learned centroid. It will be noted that, contrary to usual fuzzy clustering, the present disclosure utilizes the fuzzy clustering process iteratively to formulate the centroid of a single cluster corresponding to a quasi-stationary hemodynamic signal segment that comprises a pulses. In some exemplary embodiments, the fuzzy clustering process can be repeated with the newly learned centroid. The fuzzy clustering process can be concluded after a difference between two consecutive centroids is below a given threshold value.

In step 808, physiological parameters are determined based on the resulting learned centroid that is synthesized as the pulse best representing the pulse sequence of a segment. In some exemplary embodiments, the physiological parameters outcome comprises systolic and diastolic blood pressure, cardiac output, stroke volume heart rate, SpO2, a combination thereof, or the like.

In some exemplary embodiments, upon learning the centroid pulse shape and representing it as vectors in the N dimensional feature space, the physiological parameters values can be predicted. This can be performed by learning the mapping between pulse shape feature vector and target physiological values over time. e.g., given BP values that correspond to specific pulse shapes, thus learning the mapping between the N dimensional vectors and BP. The use of such model can indicate features that are more relevant to predicting BP.

In some exemplary embodiments, the interval between the incident pulse peak and reflected waves peaks is inversely proportional to BP, as higher BP results in faster reflected waves. For example, these parameters values can be shown in the health dashboard, as depicted in FIG. 2A.

In some exemplary embodiments, the wide population fuzzy clustering space of membership of each point in all clusters provides a space where individual pulse shape is clustered according to features. This fuzzy clustering for a population is multidimensional and used for internal computations.

In step 809, an outcome comprising the physiological parameters is displayed in forms of displays, such as display 130 shown in FIG. 3, or the like. In some exemplary embodiments, the physiological parameters can be communicated to the user or a server over the internet by Wi-Fi, Bluetooth, a combination thereof, or the like.

It should be noted that the construction of N dimensional fuzzy clustering for populations is to assist in creating a landscape of where an individual user is positioned in relation to her/his population group. The input parameters are taken both from the PPG, ECG, spectroscopy, etc. signals representing health situation in a given moment in real time, as well as background data of the user health history and answers to questionnaires. In addition to traditional BP parameters, the present disclosure also analyzes it to its basic components and causes. For example, the CHM system of the present disclosure can estimate BP total value but divide it to “Good BP” and “Bad BP” parts based on physiological model and not only regression or neural network.

Since “Good BP” resulting from increase in CO, compared to “Bad BP” resulting from increased vascular resistance, the present disclosure differentiate between Good and Bad BPs and point to the source of hypertension. i.e. if the reason for BP increase is because of increase in CO (Good BP) or because of increase in Vascular Resistance (after load) of the heart. This will help differentiate between large CO factor (Good BP) and small SVR (systemic vascular resistance) mainly because of high SVR (Bad BP) resulting from narrow clogged arteries, given that BP=CO×SVR.

According to other aspects of the present subject matter, a method and apparatus are provided to generate a personal health map for each patient that provides, at a glance, the position of the patient on the health map at any given time and track the positions on the map over time. The theoretical framework is that of fuzzy sets, where each patient gets different level of membership in different pathological and healthy conditions; i.e. nurses can track from the nurse station the personal health map of all patients in any condition of awareness and get alerts if health of any of the patients deteriorates and needs attention. The personal health map indicates the patient course during the night, as an example, and condition can be assessed at one glance in the map. Relevant health maps can also be shared on the doctor in charge smart phone. As will be shown herein after, actual patients were monitored using known methods and compared to the continuous method taught herein. Results show the efficiency of reliability of the method of the present disclosure.

In all studies performed, the tens of thousands of BP measurements with simultaneous BP pulse shapes, fuzzy clustering algorithm that were developed by the inventor of this subject matter were employed to cluster the pulse shapes in the N dimensional feature space and minimize dimensionality by looking for the smallest dimensionality space, and its 2D projections. By doing it, it was discovered that different health conditions fell into different clusters. e.g. diabetics tended to fall into a cluster of their own, where the severity of the diabetic condition indicated the position between diabetic centroid and healthy subjects.

The finding that different health condition can be defined by the BP pulse shape clustering, generates a continuous health space. Cluster centroids that represent various pulse shape belong to different health conditions. This provides a powerful tool to see patients progress or deterioration in a quantitative way that also indicates the way for more effective interventions. The invention can help with how to detect deterioration in early stage in an unobtrusive and automatic way during night watch.

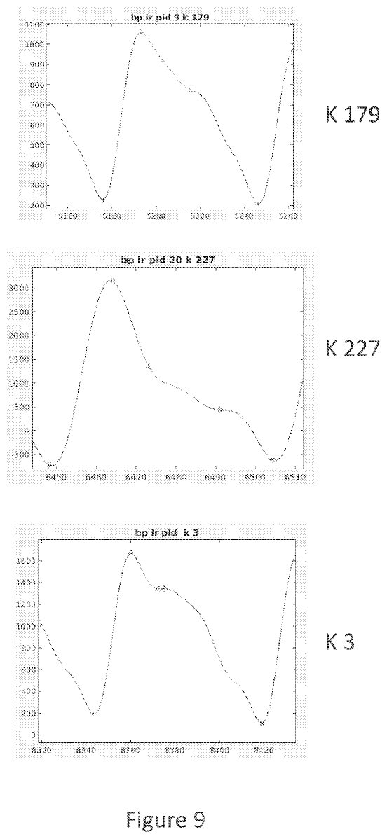

Reference is made to FIG. 9 illustrating examples of BP pulse shapes. Each centroid in a fuzzy clustering can be represented by a BP pulse shape of the examples shown herein or any other BP pulse shape. The pulse shape marked as k 179 has moderate size reflected wave and moderate interval between the forward going ejected pulse and the reflected pulse.

The second pulse shape, marked as k 227, has much smaller reflected pulse with a long interval between the forward moving pulse component (percussion) and the reflected pulse. It can be seen that the Tidal pulse in between that is generated by the flexible Aorta and is typical to younger subjects (30 years or less). Normally such pulse shape will be associated with younger and more healthy subjects. In health map that can be based on such examples of pulse shapes, this is considered to be typical to the healthy zone, as will be shown herein after.

The third pulse shape marked as k 3 shows high reflected wave closer to the forward moving pulse component (percussion). This is typical, if recorded in rest, to unhealthy subject and to a unhealthy zone of a health map as will be described herein after.

As we mention before, pulse shapes change over time and as result of interventions like life-style (exercise, sleep, healthy or unhealthy meals, relaxation etc.) or medication, or the like. The intervention can be interventions such as specific medication, specific dosage of the medication, interaction with other medications, physical rehabilitation, surgical procedure, stressful situation, life-style interventions such as exercise, sleep, healthy and unhealthy meals, glucose load, air purity, infectious disease, ionizing, and non-ionizing radiation, and the like.

In a clinical study in Calcutta hospital for hypertensive and diabetic patients, 30 hypertensive and diabetic patients were continuously monitored for 24 hours both with ABPM recording their BP and HR every 15 min and continuous PPG and SpO2, respiration for prediction of BP and cardiac output. In a second clinical trial, a comparison was made of hemodynamics prediction from a continuous hemodynamics CNAP device, during hemodialysis in Fresenius Kidney Center, in St. Louis hospital using 2-4 light wavelengths that allowed the inventors of the present subject matter also to interrogate the capillary bed at different penetration levels to estimate features related to the microcirculation.

The studies performed provided tens of thousands of BP measurements with simultaneous BP pulse shapes. Fuzzy clustering algorithms in accordance with the subject matter were employed to cluster the pulse shapes in a N dimensional feature space and minimize dimensionality by looking for the smallest dimensionality space, and its 2D projections. It was surprisingly discovered that different health conditions fell into different clusters. e.g. diabetics tended to fall into a cluster of their own, where the severity of the diabetic condition indicated the position between diabetic centroid and healthy subjects.

The finding that different health condition can be defined by the BP pulse shape clustering, generates a continuous health space. Cluster centroids that represent various pulse shape belong to different health conditions. This provides a powerful tool to observe patients progress or deterioration in a quantitative way that also indicates the way for more effective interventions. The subject matter disclosed herein can assist in detecting possible deterioration in early stage in an unobtrusive and automatic way during night watch, as an example.

The personal health navigation system according to the present subject matter uses machine learning mechanism configured to confirm with different physiological parameters of the individual.

The machine learning mechanism is configured to:
estimate blood pressure from PPG signal;
determine physiological model of pulse propagation in an arterial tree;
delineate the signal to separate pulses;
use a second derivative of the PPG signal and Gaussian features indicative of the individual physiological condition.

The machine learning mechanism configured also to:
represent feature vectors as points in an N dimensional feature space;
construct centroid points according to the health map and the individual details selected from a group of details such as gender, age, height, weight, previous BP measurements, and the like;
perform fuzzy clustering including dimensionality reduction to improve the centroid based on recorded pulses;
position each BP pulse vector in a clustering space.

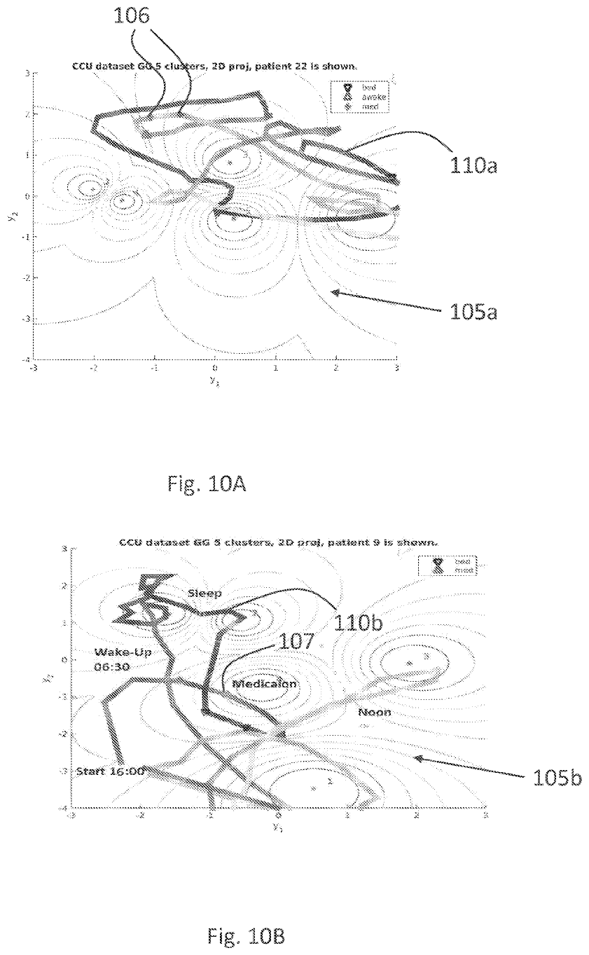

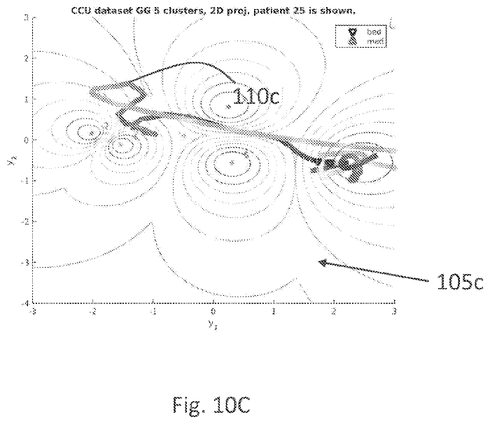

Reference is now made to FIGS. 10A-10C depicting different clusters of BP pulse shapes constructed by fuzzy clustering in a BP pulse features space to generate fuzzy clusters around centroids, in accordance with some exemplary embodiments of the disclosed subject matter. The BP pulse N features, as described herein before, describe N dimensional feature space, where each BP pulse shape is a vector in this N dimensional space. Application of fuzzy clustering that comprises also dimensionality reduction. To enable visualization, we use the most informative 2D projection. This 2D health space is created for a wide variety of individuals with great diversity of BP Pulse shapes: males and females, old and young people, so called healthy and patients that suffer from various diseases like congestive heart failure (CHF), hypertension, diabetic, chronic obstructive pulmonary disease (COPD), or similar. Also, these potential users have different profiles of physical activity, eating habits and diets, and various medications. The subjects were followed for 24 h while continuously recording their PPG (BP pulses) and their SpO2 using ambulatory BP monitor (ABPM) (that was activated every predetermined time interval (e.g. every 15 min) to measure their BP. The resulting trace in time, generated by the series of vectors corresponding to three individuals over 24 h is depicted in FIGS. 10A-10C. FIG. 10A-10C illustrates the zones or fuzzy clusters formed on a 2-dimensional health map 105a, 105b, and 105c onto which curves 110a, 110b, and 110c are formed, respectively, from actual measurements of the individual through time. The curves represents the specific health of this individual on the health maps formed from the results of the fuzzy clustering of a gross population. As mentioned herein before, different areas on the map 105a-c represent different health conditions. In the upper left side of the health map 105a-c, there is a healthy zone—fuzzy clusters of healthy people were found to be clustered in these areas. Diagonally from there, in the lower right side of the health maps 105a-c, results from non healthy people were clustered. The curves 110a-c are results taken during the hours of the day and the night from continuous monitoring of the user's hemodynamic parameters in accordance with the embodiments of the present disclosure. If can be seen that curve 110a is in the upper left side of the health map when the individual is being medicated (indicated as a plus sign+106 in the figure).

Similarly, curve 110b shows the movement of the measured parameters travelling on the health map 105b. After receipt of medication, indicated by arrow 107, the curve 110b takes a turn and goes to the healthy zone. Where the user stays for many hours during the day.

Similarly, the user shown in FIG. 10c, can be clearly seen to travel from the non healthy zone to the healthy zone after medication.

Using those health maps can assist the care taker or the user himself to navigate through the health map by changing his life style or taking appropriate medication. The immediate follow up of the status of the user through the curve on the health map assists in understanding the consequences of taking a certain medication, changing habits, changing diet, etc.

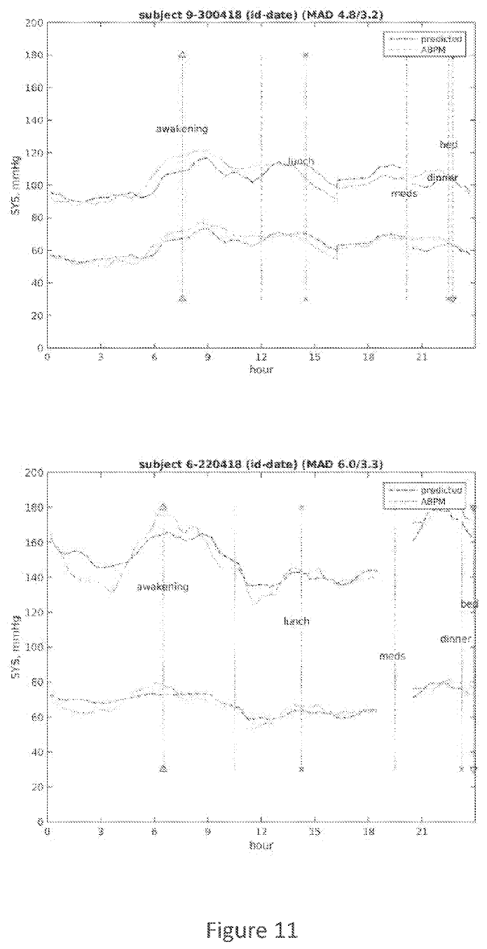

References is made to FIG. 11 depicting examples for two subjects of mapping between BP pulse shapes and BP levels, derived from the continuous PPG in accordance to an exemplary embodiment of the present subject matter. The assumption that there is a one-to-one mapping between BP Pulse shapes and BP level is probably untrue. This adjustment of the prediction algorithm based on the fuzzy clustering membership levels, leads the inventors to good tracking of the measured BP, as depicted in the figures.

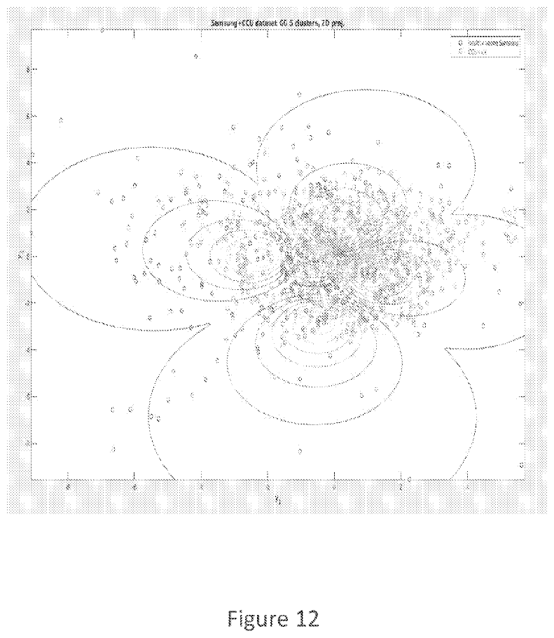

Reference is made to FIG. 12 depicting health map in accordance to an exemplary embodiment of the present subject matter. The health map illustrates both so called “Healthy” and “Sick” (Hypertensive, Diabetic . . . ) pulse shapes are shown. Blue circles designate Healthy and green pulse shapes indicate Sick. It can be seen that the distinction is somewhat arbitrary as Healthy pulse shapes fall in Sick zones and vice versa. This is understandable since hypertensive user that takes medication can have pulse shapes that look healthy. A so-called healthy population include prehypertensive and prediabetics. However, it can still be seen that the majority of the blue circles are on the left lower side and green concentrate are more on the right upper area on the map.

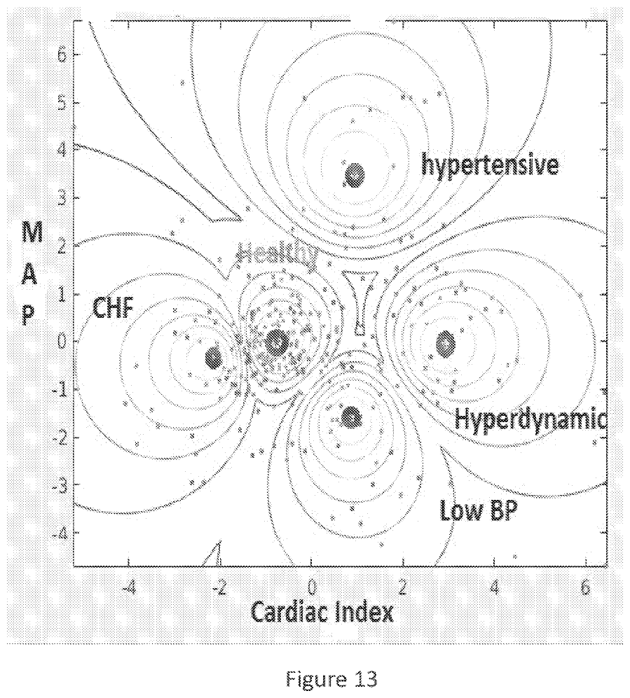

Reference is made to FIG. 13, where health space is spanned by known axes of mean arterial pressure and cardiac output. This allows, in accordance with preferred embodiments of the present subject matter to recognize zones that are typical for healthy status (substantially in the center), congestive heart failure CHF on the left-hand side of the space, and other diseases and situations. Such map allows not only to show how far an individual is from the healthy zone, but to indicate risks of specific diseases and even suggest what kind of treatment might be most appropriate for this individual.

This assumption underlying most existing algorithms for derivation of BP from BP pulse shapes, lead to their failure in predicting correctly BP level for people taking medication or over longer period of time, leading to frequent need for calibration.

The present disclosed subject matter may be a system, a method, and/or a computer program product. The computer program product may include a computer readable storage medium (or media) having computer readable program instructions thereon for causing a processor to carry out aspects of the present disclosed subject matter.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present disclosed subject matter may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present disclosed subject matter.

Aspects of the present disclosed subject matter are described herein with reference to flowchart illustrations and/or block diagrams of methods, apparatus (systems), and computer program products according to embodiments of the disclosed subject matter. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present disclosed subject matter. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The corresponding structures, materials, acts, and equivalents of all means or step plus function elements in the claims below are intended to include any structure, material, or act for performing the function in combination with other claimed elements as specifically claimed. The description of the present disclosed subject matter has been presented for purposes of illustration and description but is not intended to be exhaustive or limited to the disclosed subject matter in the form disclosed. Many modifications and variations will be apparent to those of ordinary skill in the art without departing from the scope and spirit of the disclosed subject matter. The embodiment was chosen and described in order to best explain the principles of the disclosed subject matter and the practical application, and to enable others of ordinary skill in the art to understand the disclosed subject matter for various embodiments with various modifications as are suited to the particular use contemplated.

1. A method of constructing a health map based on physiological pulse shape features space of a plurality of users, the method comprising:
sensing by at least one sensor a stream of pulses;
extracting features and forming fuzzy clusters from the features; and
constructing the health map by 2-dimensional projection of n-dimensional space.

2. The method according to claim 1, wherein a position of a specific pulse shape of a user at a specific time, indicates the health status of the user at this point in time.

3. The method according to claim 1, wherein a plurality of positions of pulse shapes of one of the plurality of users over time generates a trace that indicates changes of health status over time.

4. The method according to claim 3, wherein the positions of pulse shapes of the one of the plurality of users over time generates the trace that indicates changes over time and indicates interventions.

5. The method according to claim 4, wherein the interventions are selected from interventions consisting of medication, dosage of medication, interaction with other medications, physical rehabilitation, surgical procedure, stressful situation, exercise, sleep, healthy and unhealthy meals, glucose load, air purity, infectious disease, ionizing and non-ionizing radiation, and exposure to toxins.

6. The method according to claim 2, wherein the health status is selected from a group of health statuses consisting of healthy, having health disorders, not healthy, male, female, old, young, having congestive heart failure (CHF), hypertensive, diabetic, having chronic obstructive pulmonary disease (COPD), having blood analytes disorder, electrical activity disorder, combinations of the above and the like.

7. The method according to claim 1, wherein the method further comprises:
sensing by the at least one sensor a stream of pulses of an individual;
extracting features by decomposing sensed pulses of the individual;
classifying fuzzy clusters of the features;
estimating physiological parameters of the individual; and
placing the physiological parameters over time on the health map to form a personal health navigation system.

8. A personal health navigation system for an individual comprising:
a health map provided as in claim 1; and
recommended paths from a user's position to a target zone on the health map.

9. The personal health navigation system according to claim 8, wherein the health map comprises fuzzy clusters that are indicative of physiological parameters and therefore form areas on the health map that are indicative of health status.

10. The personal health navigation system according to claim 9, wherein the health status is selected from a group of health statuses consisting of healthy, sick, not healthy, male, female, old, young, having congestive heart failure (CHF), having hypertension, having diabetic, having chronic obstructive pulmonary disease (COPD), and the like.

11. The personal health navigation system according to claim 7, wherein placing health parameters of the individual on the health map provides indication of a position of the individual on the map.

12. The personal health navigation system according to claim 7, wherein the health map is provided with indications of healthy and unhealthy zones according to pulse features.

13. The personal health navigation system according to claim 7, wherein the navigation system further comprises treatment paths indicated on the health map.

14. The personal health navigation system according to claim 7, wherein the health map further comprises machine learning mechanism configured to confirm with different physiological parameters of the individual.

15. The personal health navigation system according to claim 14, wherein the machine learning mechanism is configured to:
estimate blood pressure from PPG signal;
determine physiological model of pulse propagation in an arterial tree;
delineate the signal to separate pulses; and
use a second derivative of the PPG signal and Gaussian features indicative of the individual physiological condition.

16. The personal health navigation system according to claim 14, wherein the machine learning mechanism is configured to:
represent feature vectors as points in an N dimensional feature space;
construct centroid points according to the health map and the individual details selected from a group of details consisting of gender, age, height, weight, previous BP measurements, and the like;
perform fuzzy clustering including dimensionality reduction to improve the centroid based on recorded pulses; and
position each BP pulse vector in a clustering space.
